(19) United States
(12) Patent Application Publication
(10) Pub. No.: US 2024/0168024 A1
(43) Pub. Date: May 23, 2024

(54) METHOD AND SYSTEM FOR DIAGNOSING WHETHER AN INDIVIDUAL HAS LUNG CANCER

(71) Applicant: Hangzhou Guangkeande Biotechnology Co., Ltd., Zhejiang (CN)

(72) Inventors: Junli GAO, Zhejiang (CN); Junshun GAO, Zhejiang (CN); Xiaojun PENG, Zhejiang (CN); Weixin WANG, Zhejiang (CN); Qinqin LOU, Zhejiang (CN); Hong GUAN, Zhejiang (CN)

(21) Appl. No.: 18/457,010

(22) Filed: Aug. 28, 2023

(30) Foreign Application Priority Data
Nov. 22, 2022 (CN) 202211486610.8

(51) Int. Cl. G01N 33/574; G16B 25/10; G16B 40/20; G16H 50/20

(52) U.S. Cl. CPC G01N 33/57423; G16B 25/10; G16B 40/20; G16H 50/20

(57) ABSTRACT

The present disclosure provides a biomarker for detecting lung cancer and use thereof. A proteomics method is used to analyze a protein with significant differences in blood of a patient with lung cancer and normal people, such that a series of biomarkers capable of early predicting an occurrence risk of lung cancer are screened out, a group of biomarkers are further screened to construct a diagnosis model for lung cancer, and the model may be used for conveniently, non-invasively and effectively predicting whether an individual suffers from lung cancer or not, and meets clinical needs.

### CROSS-REFERENCE TO RELATED APPLICATIONS

The present application claims the priority of the Chinese patent application with an application No. 202211486610.8 on Nov. 22, 2022. The abstract, description, claims, and drawings of the description of the present application are used in its entirety by the present application.

### INCORPORATION-BY-REFERENCE OF SEQUENCE LISTING

This application includes an electronically submitted sequence listing in .xml format. The .xml file contains a sequence listing entitled 2023-08-28-sqlist.xml created on Aug. 28, 2023 and is 7,353 bytes in size. The sequence listing contained in this .xml file is part of the specification and is hereby incorporated by reference herein in its entirety.

### BACKGROUND OF THE INVENTION

### Field of the Invention

The present disclosure relates to the field of medicine, specifically, use of proteomics to screen a biomarker for lung cancer and use of the biomarker in diagnosing lung cancer, particularly a biomarker for predicting an occurrence risk of lung cancer and use thereof.

### Description of the Related Art

Proteomics is a scientific field dedicated to investigating the composition, location, changes, and interactions within cells, tissues, and organisms. It encompasses the study of protein expression patterns and functional profiles. The emergence of liquid chromatography-mass spectrometry (LC-MS/MS), facilitated by advancements in mass spectrometry technology, has greatly contributed to proteomics research. LC-MS/MS has become a crucial tool in this field. The development of proteomics carries significant importance in various areas, such as the search for disease diagnostic markers, drug target screening, toxicology research, and more. As a result, it finds wide application in medical research.

Lung cancer is one of the most common malignant tumors in clinics, with a high degree of malignancy and a rapid course of disease. Its prevalence and mortality rates rank first among malignant tumors, showing a rising trend year by year. The data published by the National Health Commission shows that lung cancer is a leading cause of death from malignant tumors in China, and accounts for 20% or more of all malignant tumors.

An accurate diagnosis of lung cancer is key to reducing mortality, but currently, no effective diagnostic method is available. 70% or more of patients with lung cancer have missed an optimal treatment opportunity when diagnosed. At present, there are mainly two methods of histology and imaging for diagnosing lung cancer. But the two methods have certain limitations. Since immunology and molecular biology develop, a tumor-associated protein marker shows more and more important clinical value in diagnosis and treatment of lung cancer, and has become an indispensable biological indicator for auxiliary diagnosis, observation of efficacy, and judgment of prognosis.

A plurality of tumor markers for the diagnosis of lung cancer, pathological typing, clinical staging, and judgment of prognosis and efficacy have been found clinically, but the diagnosis efficiency of the currently common markers (CEA and CA125) for lung cancer is not ideal. A specific tumor marker has not been found to have a higher sensitivity and specificity to diagnosis of lung cancer.

Therefore, it is of important clinical value to find a new related marker for diagnosis of lung cancer, combine a plurality of markers, and use a suitable prediction model for diagnosis of lung cancer.

### BRIEF SUMMARY OF THE INVENTION

Aiming at the problems existing in the prior art, the present disclosure provides a biomarker for detecting lung cancer. A proteomics method is used to analyze a protein with a significant difference in blood of a patient with lung cancer and normal people, such that a series of new biomarkers capable of early predicting an occurrence risk of lung cancer are screened out, a group of biomarkers are further screened to construct a diagnosis model for lung cancer, and the model may be used for conveniently, non-invasively and effectively predicting whether an individual suffers from lung cancer or not, and meets clinical needs.

In one aspect, the present invention provides use of a biomarker in preparing a reagent for predicting whether an individual has lung cancer or not. The biomarker is selected from one or more of the following: Piggy Bac transposable element-derived protein 5 (PGBD5), cathepsin G (CTSG), tryptophanyl-tRNA synthetase 1 (WARS1), L-selectin (SELL), and pro-surfactant protein B (Pro-SFTPB).

Through a TMT labeled quantified proteomics research, an ultra-performance liquid chromatography-tandem mass spectrometry (LC-MS/MS) is used to analyze blood samples of a healthy group and a lung cancer patient group. Proteins with significant differences between a lung cancer sample and a control sample are determined by orthogonal partial least squares. Finally, 5 new proteins related to lung cancer are obtained as biomarkers for efficiently predicting whether an individual has lung cancer or not.

In some embodiments, the biomarker for predicting whether an individual has lung cancer or not may be a detection target to prepare a detection reagent, such as a sample pretreatment reagent, an antigen or an antibody, and other biological reagents and kits suitable for detecting the biomarker; and a standardized reagent or a kit and the like may also be developed to be suitable for detecting the biomarker by LC-UV or LC-MS.

In some embodiments, the Piggy Bac transposable element-derived protein 5 (PGBD5) is a protein or an amino acid sequence with a UniProt database number of Q8N414; the cathepsin G (CTSG) is a protein or an amino acid sequence with a UniProt database number of P08311; the tryptophanyl-tRNA synthetase 1 (WARS1) is a protein or an amino acid sequence with a UniProt database number of P23381; the L-selectin (SELL) is a protein or an amino acid sequence with a UniProt database number of P14151; and the pro-surfactant protein B (Pro-SFTPB) is a protein or an amino acid sequence with a UniProt database number of P07988.

Further, the biomarker comprises PGBD5, CTSG, WARS1, SELL, and Pro-SFTPB.

In some embodiments, the biomarker comprises the PiggyBac transposable element-derived protein 5 (PGBD5), the cathepsin G (CTSG), the tryptophanyl-tRNA synthetase 1 (WARS1), the L-selectin (SELL), cytokeratin 19 fragment (Cyfra21-1), carcinoembryonic antigen (CEA), cancer antigen 125 (CA125), and the pro-surfactant protein B (Pro-SFTPB).

Furthermore, the reagent is used for detecting the biomarker in a fluid sample. The fluid sample comprises any one of blood, urine, saliva, and sweat.

In some embodiments, the biomarker of the present disclosure is obtained by screening a blood sample, and is particularly suitable for being developed into a blood detection reagent or a kit for predicting lung cancer.

In the present disclosure, biomarkers for lung cancer are screened from blood; the biomarkers are significantly different in the blood of a patient with lung cancer and a patient without lung cancer. By collecting the blood samples, the biomarkers in the blood of an individual may be detected to predict or auxiliary diagnose whether the individual has lung cancer or not or has a possibility of suffering from lung cancer, or the biomarkers in the blood of a certain group may be detected to classify the group into a lung cancer group or a non-lung cancer group.

Furthermore, the detection of the biomarker in the fluid sample is to detect the presence or relative abundance or concentration of the biomarker in the fluid sample of the individual.

In some embodiments, the relative abundance is preferably used and a peak area of the biomarker in a detection spectrum is obtained by ultra-performance liquid chromatography-tandem mass spectrometry. For example, if the average peak area of a biomarker in a control sample (an individual not suffering from lung cancer) is 500 and the average peak area measured in lung cancer sample is 3,000, the abundance of the biomarker in the lung cancer sample is considered to be 6-fold that in the control sample.

In the other aspect, the present disclosure provides a biomarker combination for predicting whether an individual has lung cancer. The biomarker comprises a combination selected from the following two or more biomarkers: PGBD5, CTSG, WARS1, SELL, Cyfra21-1, CEA, CA125, and Pro-SFTPB.

Furthermore, the biomarker comprises the PGBD5, the CTSG, the WARS1, the SELL, the Cyfra21-1, the CEA, the CA125, and the Pro-SFTPB.

The detected data of clinical lung cancer samples show that the AUC value may reach 0.916 by only using the 8 biomarkers to predict lung cancer, and the effect is obviously better than that of an existing multi-biomarker combined prediction model for lung cancer.

In the other aspect, the present disclosure provides a kit for predicting whether an individual has lung cancer or not. The kit comprises the biomarkers or a detection reagent of the biomarker combination.

In some embodiments, the detection reagent is an antibody of the biomarker, and the antibody is a monoclonal antibody.

In another aspect, the present disclosure provides a system for predicting whether an individual has lung cancer or not, wherein the system comprises a data analysis module, the data analysis module is used for analyzing a detection value of a biomarker, and the biomarker is selected from the following one or more: PGBD5, CTSG, WARS1, SELL, and Pro SFTPB; or selected from a combination of the following any two or more biomarkers: the PGBD5, the CTSG, the WARS1, the SELL, Cyfra21-1, CEA, CA125, and the Pro-SFTPB.

Furthermore, the biomarker comprises the PGBD5, the CTSG, the WARS1, the SELL, the Cyfra21-1, the CEA, the CA125, and the Pro-SFTPB.

Furthermore, the data analysis module evaluates whether an individual has lung cancer or not by substituting the detection value of the biomarker into an equation and calculating a predictive value that predicts whether the individual has lung cancer or not, and the equation is as follows:

Y=Σᵢ₌₁^{m}Kᵢ*Xᵢ+b
wherein Y is a predictive value, i represents an i^{th} biomarker, m represents the number of biomarkers (m=8), Xᵢ represents a detection value of the i^{th} biomarker (μg/mL), Kᵢ represents a coefficient of the i^{th} biomarker, and b is a constant 3.261652; and
the coefficient Kᵢ is shown in the following table:

| Biomarker | Coefficient |
|---|---|
| Cyfra21-1 | −0.76761 |
| CEA | 1 |
| CA125 | 0.434921 |
| Pro-SFTPB | −0.72697 |
| PGBD5 | −0.14199 |
| CTSG | 1 |
| WARS1 | 1 |
| SELL | 1 |

In some embodiments, when the predicted value Y is less than or equal to 0.734, it is determined that the individual is not a lung cancer patient; when the predicted value Y is greater than 0.734, it is determined that the individual is a lung cancer patient.

In some embodiments, the system further comprises a data detection system, and a data input and output interface; the data detection system is used to detect a biomarker in a sample and obtain a detection value; and an input interface in the data input and output interface is used to input the detection value of the biomarker, after the data analysis module analyses the detection value, an output interface is used to output an analysis result of whether an individual has lung cancer or not, for example, the output interface is a display or a printing module that prints a result.

In the other aspect, the present disclosure provides a method for diagnosing whether an individual has lung cancer or not, wherein the method comprises: providing a fluid sample from an individual, testing a concentration of a biomarker in the fluid sample, and distinguishing the individual into a healthy individual and an individual suffering from lung cancer according to a concentration, wherein the biomarker is selected from one or more of the following: PGBD5, CTSG, WARS1, and SELL.

In some embodiments, the biomarker comprises PGBD5, CTSG, WARS1, and SELL.

In some embodiments, the fluid sample comprises any one of blood, urine, saliva, and sweat.

In some embodiments, the fluid sample is a blood sample or a serum sample.

In some embodiments, a measuring method comprises an enzyme-linked immunosorbent assay (ELISA), a protein/peptide fragment chip detection, an immunoblotting, a microbead immunoassay or a microfluidic immunoassay.

In some embodiments, the biomarker further comprises Cyfra21-1, CEA, CA125, and Pro-SFTPB, and the marker comprises a combination of two or more selected from the following biomarkers: the PGBD5, the CTSG, the WARS1, the SELL, the Cyfra21-1, the CEA, the CA125, and the Pro-SFTPB.

In some embodiments, the biomarker is a combination of three or more of the following biomarkers: the PGBD5, the CTSG, the WARS1, the SELL, the Cyfra21-1, the CEA, the CA125, and the Pro-SFTPB.

In some embodiments, the biomarker is a combination of the following eight biomarkers: the PGBD5, the CTSG, the WARS1, the SELL, the Cyfra21-1, the CEA, the CA125, and the Pro-SFTPB.

In some embodiments, the biomarker consists of the following markers: the PGBD5, the CTSG, the WARS1, the SELL, the Cyfra21-1, the CEA, the CA125, and the Pro-SFTPB.

In some embodiments, the method further comprises a data analysis module and the data analysis module is used to input a concentration value of a biomarker for analysis.

In some embodiments, the data analysis module evaluates whether an individual has lung cancer or not by substituting the concentration value of the biomarker into an equation and calculating a predictive value that predicts whether the individual has lung cancer or not, and the equation is as follows:

Y=Σᵢ₌₁^{m}Kᵢ*Xᵢ+b
wherein Y is a predictive value, i represents an i^{th} biomarker, m represents the number of biomarkers (m=8), Xᵢ represents a concentration value of the i^{th} biomarker, Kᵢ represents a coefficient of the i^{th} biomarker, and b is a constant 3.261652; and
the coefficient Kᵢ is as shown in the following table:

| Biomarker | Coefficient |
|---|---|
| Cyfra21-1 | −0.76761 |
| CEA | 1 |
| CA125 | 0.434921 |
| Pro-SFTPB | −0.72697 |
| PGBD5 | −0.14199 |
| CTSG | 1 |
| WARS1 | 1 |
| SELL | 1 |

In some embodiments, when the predicted value Y is less than or equal to 0.734, it is determined that the individual is not a lung cancer patient; when the predicted value Y is greater than 0.734, it is determined that the individual is a lung cancer patient.

In some embodiments, the PGBD5 is an amino acid sequence with a UniProt database number of Q8N414; the CTSG is an amino acid sequence with a UniProt database number of P08311; the WARS1 is an amino acid sequence with a UniProt database number of P23381; the SELL is an amino acid sequence with a UniProt database number of P14151; the Pro-SFTPB is an amino acid sequence with a UniProt database number of P07988; the CA125 is an amino acid sequence with a UniProt database number of Q8WXI7; the CEA is an amino acid sequence with a UniProt database number of Q13984; and the Cyfra21-1 is an amino acid sequence with a UniProt database number of P08727.

In another aspect, the present disclosure provides the use of the system in constructing a detection model of a probability value for predicting whether an individual has lung cancer or not.

The present disclosure has the following beneficial effects:

1. 5 new biomarkers, PGBD5, CTSG, WARS1, SELL, and Pro-SFTPB, capable of predicting an occurrence risk of lung cancer early are screened; and

2. Different biomarkers are respectively used to construct a diagnosis model of lung cancer, and it is found that a diagnosis model for lung cancer constructed by 8 biomarkers including PGBD5, CTSG, WARS1, SELL, Cyfra21-1, CEA, CA125, and Pro-SFTPB is optimal, may be used for more efficiently predicting whether an individual suffers from lung cancer or not, and has an AUC value reaching 0.916, and an effect obviously better than that of an existing diagnosis model of lung cancer.

### BRIEF DESCRIPTION OF THE DRAWINGS

FIG. 1 shows a Wilcoxon result of two groups of healthy control and lung cancer in example 1;

FIG. 2 shows the analysis results of ROC and OPLS-DA of the two groups of healthy control and lung cancer in example 1;

FIG. 3 shows an AUC result of models constructed under different hyper-parameter combinations by a glmnet algorithm in example 3;

FIG. 4 shows a ROC curve in a model group of lung cancer combined diagnosis model constructed in example 3;

FIG. 5 shows a ROC curve in a test group of the lung cancer combined diagnosis model constructed in example 3;

FIG. 6 shows a result of a performance evaluation in the test group of the lung cancer combined diagnosis model constructed in example 3; and

FIG. 7 shows ROC curves of different lung cancer diagnosis models constructed in example 3.

### DETAILED DESCRIPTION OF THE INVENTION

### (1) Diagnosis or Detection

Diagnosis or detection herein refers to detecting or assaying a biomarker in a sample, or the content, such as the absolute content or the relative content, of a target biomarker, and then indicating whether an individual providing a sample may have or suffer from a disease, or have a possibility of a disease, by the presence or the amount of the target marker. Meanings of the diagnosis and the detection herein may be interchanged. A result of the detection or the diagnosis may not be directly used as a direct result of the disease, but an intermediate result. If a direct result is obtained, whether an individual suffers from a disease may only be confirmed through other auxiliary means such as pathology or anatomy. For example, the present disclosure provides a plurality of new biomarkers correlated with lung cancer. Changes in the content of the markers are directly correlated with whether an individual has lung cancer or not.

### (2) Correlation of Marker or Biomarker With Lung Cancer

A marker and a biomarker have the same meaning in the present disclosure. A correlation here means that the presence or amount change of a biomarker in a sample is directly correlated with a particular disease, e.g. a relative increase or decrease of the amount indicates that a possibility of an individual suffering from the disease is higher than that of a healthy person.

If multiple different markers are present in a sample simultaneously or in relatively varying content, an individual also has a higher possibility of suffering from the disease than a healthy person. That is, some markers in the marker species are strongly correlated with a disease, some markers are weakly correlated with a disease, or some markers are not even correlated with a specific disease. One or more of the markers with a strong correlation may be used as a marker for diagnosing a disease. The markers with a weak relevance may be combined with the strong markers to diagnose a certain disease, so as to increase the accuracy of a detection result.

With regard to a plurality of biomarkers in serum found in the present disclosure, these markers may be used to distinguish a patient with lung cancer from a healthy person. The markers herein may be used alone as an individual marker for a direct detection or diagnosis. Such markers are selected to indicate that relative changes in the content of the markers are strongly correlated with lung cancer. Of course, it may be understood that simultaneous detection of one or more markers strongly correlated with lung cancer may be selected. It is normally understood that in some embodiments, a selection of strongly correlated biomarkers for detection or diagnosis may achieve a certain standard of the accuracy, for example, 60%, 65%, 70%, 80%, 85%, 90%, or 95% of accuracy, which may indicate that the markers may obtain an intermediate value for diagnosing a disease, but does not indicate that an individual may be directly confirmed to suffer from a disease.

Of course, a differential protein having a larger ROC value may be selected as a diagnostic marker. The so-called strong and weak are generally calculated and confirmed by some algorithms such as a contribution rate or a weight analysis of a marker and lung cancer. Such calculation methods may be a significance analysis (p value or FDR value) and a fold change. A multivariate statistical analysis mainly comprises a principal component analysis (PCA), a partial least squares discriminant analysis (PLS-DA), and an orthogonal partial least squares discriminant analysis (OPLS-DA), and other methods such as ROC analysis, etc. Of course, other model prediction methods are possible. In a specific selection of biomarkers, differential proteins disclosed herein may be selected. Or a prediction may be performed by a model method, either by selection or in combination with other previously known marker combinations.

### DETAILED DESCRIPTION OF EMBODIMENTS

The present disclosure is further described in detail below with reference to the accompanying drawings and examples. It should be pointed out that the following examples are intended to facilitate the understanding of the present disclosure without any limitation. The reagents used in the examples are known and commercially available.

### Example 1: Screening Biomarkers of Lung Cancer by Proteomics

### 1. Sample Collection

85 cases of lung cancer and 46 cases of healthy controls were collected by the study group from August 2019 to December 2019. All enrolled patients signed an informed consent. All the patients with lung cancer were confirmed with living tissues subjected to a pathological examination, and the healthy controls were normal in a conventional physical examination. Inclusion criteria for a patient with lung cancer were: (a) no history of other malignant tumors, (b) an operation treatment within one month after a blood collection, and lung cancer confirmed by a postoperative pathological examination. The healthy persons in the control group were selected from a physical examination center. These individuals were confirmed by a chest X-ray or a thin-slice computed tomography to have no lung nodules and no history of malignant tumors. After the informed consent, all the collected serum samples were stored in a serum bank at −80° ° C.

### 2. Sample Treatment and Enzymatic Digestion

Firstly, a plasma sample was centrifuged in a centrifuge for 15 minutes (15,000×g), and a supernatant was taken, filtered, and subjected to immunoaffinity chromatography to elute 14 highly abundant proteins. Then eluate was concentrated on a centrifuge (4,000×g, 1 hour) using a concentration tube with a cut-off molecular weight of 3 kDa. A concentrate was recovered and subjected to a buffer exchange using a desalting column having a cut-off molecular weight of 7 kDa on a centrifuge (1,000×g, 2 minutes), wherein the buffer solution was AEX-A (20 mM Tris, 4 M Urea, 3% isopanopanol, and pH 8.0). A protein concentration in the sample was determined using a BCA method with the AEX-A as a blank. According to the sample grouping in Table 1, TCEP was added to the sample and the sample was incubated at 37° C. for 30 minutes for protein reduction. Then a corresponding 6-plex TMT reagent was added, and the sample was incubated at room temperature for 1 hour in a dark place to conduct a TMT labeling reaction. Thereafter, the sample was subjected to a buffer exchange using a Zeba column, wherein the exchange buffer was AEX-A. After the 6-plex TMT labeled sample was mixed, 2 mL of the AEX-A was added to the mixed samples to a final volume of 5.5 mL. The sample was filtered using a 0.22-m filter and the 6-plex TMT labeled sample was separated using a 2D-HPLC system. The collected fraction was freeze-dried. Finally Trypsin/Lys-C protease mix was added, the sample was incubated at 37° C. for 5 hours for an enzyme digestion, and 5 μL of 10% TFA was added to terminate the enzyme digestion. A total of 60 enzymatically digested 2D-HPLC fractions were used for a nano-LC-MS/MS analysis.

TABLE 1
Sample grouping for proteomics research

| Sample No. | Sample grouping | TMT-6plex |
|---|---|---|
| Control 1 | Control | 126 |
| Control 2 | Control | 127 |
| Control 3 | Control | 128 |
| Case 1 | Case | 129 |
| Case 2 | Case | 130 |
| Case 3 | Case | 131 |

### 3. LC-MS/MS Data Acquisition and Library Search Analysis

An LC-MS/MS system was a combination of Easy-nLC 1200 and Q Exactive HFX, wherein a mobile phase A was an aqueous solution containing 0.1% formic acid and 2% acetonitrile, and a mobile phase B was an aqueous solution containing 0.1% formic acid and 80% acetonitrile. A self-made analysis column had a length of 20 cm, and a packing was a ReProSil-Pur C18, 1.9 μm particle from Dr. Maisch GmbH. 1 μg of a peptide fragment was dissolved by the mobile phase A and then separated by an EASY-nLC 1200 ultra-performance liquid phase system. A liquid phase gradient was set as: 0-26 min, 7%-22% B; 26-34 min, 22%-32% B; 34-37 min, 32%-80% B; and 37-40 min, 80% B, wherein a flow rate of the liquid phase was maintained at 450 nL/min.

The peptide segment separated by the high-performance liquid system was injected into a NanoFlex ion source for atomization, and then subjected to a Q active HF-X mass spectrometry. The ion source had a voltage of 2.1 kV, a first-order mass spectrometry scanning range was set to be 400-1,200, and a resolution ratio was 60,000 (MS resolution); and a secondary mass spectrometry scanning range started at 100 m/z and the resolution ratio was set at 15,000 (MS2 resolution). MS data acquisition mode was set to data-dependent acquisition (DDA) mode. The TOP 20 precursor ions sequentially enter the HCD collision cell for fragmentation and then subjected to a secondary mass spectrometry. Automatic gain control (AGC) was set at 5E4, a signal threshold was set at 1E4, and a maximum injection time was set at 22 ms. To avoid repeated scanning of a highly abundant peptide fragment, the dynamic exclusion time for a tandem mass spectrometry was set at 30 seconds.

Mass spectral data obtained by LC-MS/MS were retrieved using MaxQuant (v1.6.15.0). The data type was ion-quantified TMT proteomics data based on a secondary reporter, and a secondary spectrogram for quantification requires that parent ions in a primary spectrogram account for more than 75%. Database source: Homo_sapiens_9606_proteome of Uniprot database (release: Oct. 14, 2021, sequence: 20614). Besides, a common pollution library was added into the database, and a pollution protein was deleted during data analysis; an enzyme cutting mode was set as Trypsin/P; the number of missed cutting sites was set to be 2; a mass error tolerance of the parent ions of the First search and the Main search was respectively set to be 20 ppm and 5 ppm, and a mass error tolerance of secondary fragment ions was 20 ppm. A fixed modification was cysteine alkylation and a variable modification was the oxidation of methionine and acetylation of an N-terminal of a protein. The FDR of protein identification and PSM identification was set to be 1%.

### 4. Sample Grouping by Utilizing Orthogonal Partial Least Square Discriminant Analysis, Significance Analysis Combining, and Differential Protein Screening

Differential proteins were screened by using a mode of combining a univariate analysis and a multivariate statistical analysis, wherein the univariate analysis mainly comprises a significance analysis (p value or FDR value) and a fold change of characteristic ions in different groups, and the multivariate statistical analysis mainly comprises a principal component analysis (PCA), a partial least squares discriminant analysis (PLS-DA), and an orthogonal partial least squares discriminant analysis (OPLS-DA).

We have found 1,256 protein substances in total, including some newly discovered markers related to lung cancer, and some known and confirmed markers related with lung cancer (e.g., carcinoembryonic antigen (CEA), cancer antigen 125 (CA125), etc.).

Aiming at the found 1,256 protein substances, the protein substances with a remarkable content difference were analyzed. All statistical analyses were finished using R and specific R-related information was shown in Table 2.

TABLE 2
R and related information thereof used in the present disclosure

| Name | Version |
|---|---|
| R | 3.4.1 |
| Rstudio | 1.4.1717 |
| MixOmics | 6.10.9 |
| Ropls | 1.18.1 |

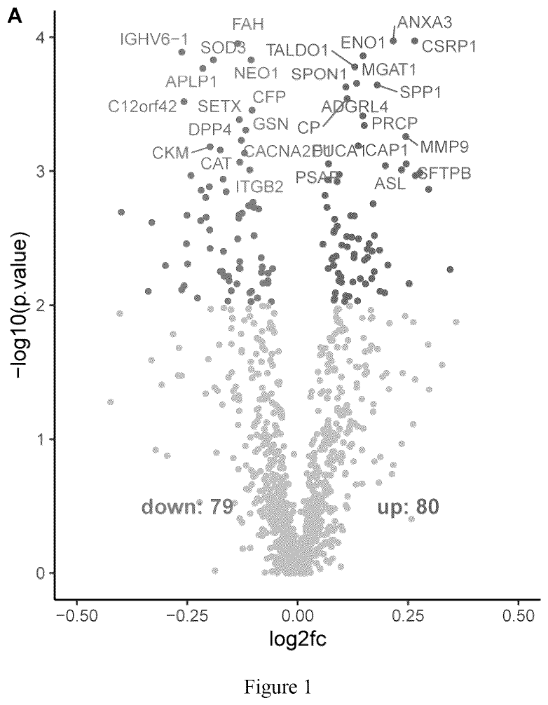

Variable importance for the projection (VIP) was calculated to measure the influence strength and the interpretation ability of an expression pattern of each protein for classification and discrimination of each group of samples. A corrected p value (FDR) was further obtained by a Wilcoxon rank sum test. A Wilcoxon rank result is shown in FIG. 1. It is found that 79 total proteins among 1,256 proteins were significantly decreased in the serum of a patient with lung cancer, and 80 proteins were significantly increased in serum of a patient with lung cancer (see FIG. 1 for details).

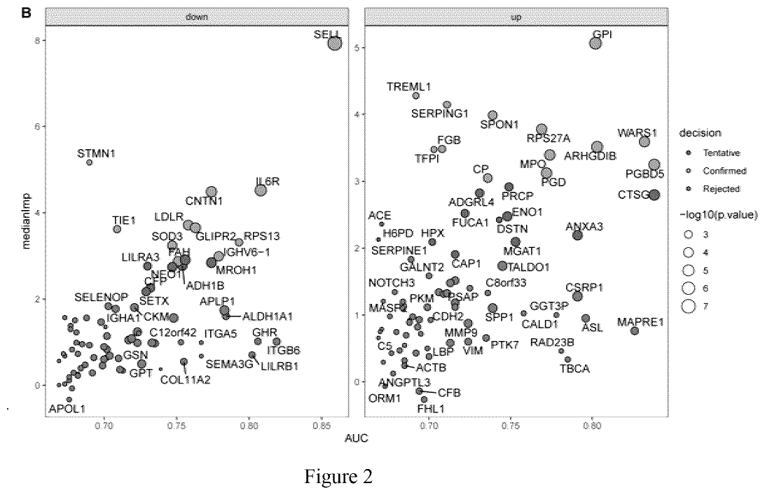

ROC and OPLS-DA analysis results are shown in FIG. 2, wherein an x-coordinate was AUC obtained by a ROC analysis, a y-coordinate was a VIP value obtained by an OPLS-DA analysis, a size of a dot represented a p value calculated by the Wilcoxon test, and a color of the dot represented a significance evaluation of the VIP value.

According to screening criteria of differential proteins: (1) VIP>1; and (2) FDR<0.05, that is, VIP>1 or FDR<0.05, a protein was determined to be significantly different between two groups, and the protein was a differential protein between the two groups. According to the screening criteria, 8 more significant differential proteins were found in total, including some new biomarkers (e.g., PiggyBac transposable element-derived protein 5 (PGBD5), cathepsin G (CTSG), tryptophanyl-tRNA synthetase 1 (WARS1), and L-selectin (SELL), and some known biomarkers for lung cancer (e.g., carcinoembryonic antigen (CEA) and cancer antigen 125 (CA 125)).

8 main significant differential proteins found in the present disclosure were shown in Table 3:

TABLE 3
Differential markers of patients with
lung cancer and normal healthy person

| | | | Number of |
|---|---|---|---|
| Name of biomarker | FDR | VIP | UniProt database |
| PiggyBac | 1.33e−5 | 3.25 | Q8N414 |
| transposable element- | | | |
| derived protein 5 (PGBD5) | | | |
| Cathepsin G (CTSG) | 2.37e−5 | 2.8 | P08311 |
| Tryptophanyl-tRNA | 3.51e−5 | 3.59 | P23381 |
| synthetase 1 (WARS1) | | | |
| L-selectin (SELL) | 3.3e−8 | 7.94 | P14151 |
| Pro-surfactant | 8.83e−4 | 1.51 | P07988 |
| protein B (Pro-SFTPB) | | | |
| Cytokeratin 19 | 3.29e−6 | 5.07 | P08727 |
| fragment (Cyfra21-1) | | | |
| Carcinoembryonic | 6.85e−06 | 4.33 | Q13984 |
| antigen (CEA) | | | |
| Cancer antigen 125 (CA125) | 2.69e−05 | 4.22 | Q8WXI7 |

The smaller FDR value and/or the larger VIP value in Table 3, to some extent, indicate that the difference in the differential compound between the two groups was more significant and that the differential compound may have a higher diagnostic value.

According to Table 3, among the 1,256 substances in serums of a patient with lung cancer and a normal healthy person, 8 differential proteins were found. The difference was more significant between the lung cancer group and the non-lung cancer group, including 5 new markers capable of efficiently predicting lung cancer: PiggyBac transposable element-derived protein 5 (PGBD5), cathepsin G (CTSG), tryptophanyl-tRNA synthetase 1 (WARS1), L-selectin (SELL), and pro-surfactant protein B (Pro-SFTPB), and 3 known biomarkers for lung cancer: carcinoembryonic antigen (CEA), cancer antigen 125 (CA 125), and cytokeratin 19 fragment (Cyfra21-1). Meanwhile, it is also verified that the known biomarkers for lung cancer had a good performance in predicting lung cancer. The L-selectin (SELL) was the most significant protein in distinguishing a patient with lung cancer from a healthy control, followed by the cytokeratin 19 fragment (Cyfra21-1), the carcinoembryonic antigen (CEA), the tryptophanyl-tRNA synthetase 1 (WARS1), and then the cathepsin G (CTSG), the PiggyBac transposable element-derived protein 5 (PGBD5), the cancer antigen 125 (CA125), and the pro-surfactant protein B (Pro-SFTPB) in sequence.

It was confirmed that the PiggyBac transposable element-derived protein 5 (PGBD5) is a protein or an amino acid sequence with a UniProt database number of Q8N414; the cathepsin G (CTSG) is a protein or an amino acid sequence with a UniProt database number of P08311; the tryptophanyl-tRNA synthetase 1 (WARS1) is a protein or an amino acid sequence with a UniProt database number of P23381; the L-selectin (SELL) is a protein or an amino acid sequence with a UniProt database number of P14151; and the pro-surfactant protein B (Pro-SFTPB) is a protein or an amino acid sequence with a UniProt database number of P07988.

The PGBD5 (Q8N414) has an amino acid sequence as follows (SEQ ID NO: 1):
MAEGGGGARRRAPALLEAARARYESLHISDDVFGESGPDSGGNPFYSTSAASRSSSAASSDDE
REPPGPPGAAPPPPRAPDAQEPEEDEAGAGWSAALRDRPPPRFEDTGGPTRKMPPSASAVDFFQL
FVPDNVLKNMVVQTNMYAKKFQERFGSDGAWVEVTLTEMKAFLGYMISTSISHCESVLSIWSG
GFYSNRSLALVMSQARFEKILKYFHVVAFRSSQTTHGLYKVQPFLDSLQNSFDSAFRPSQTQVLH
EPLIDEDPVFIATCTERELRKRKKRKFSLWVRQCSSTGFIIQIYVHLKEGGGPDGLDALKNKPQLH
SMVARSLCRNAAGKNYIIFTGPSITSLTLFEEFEKQGIYCCGLLRARKSDCTGLPLSMLTNPATPPA
RGQYQIKMKGNMSLICWYNKGHFRFLTNAYSPVQQGVIIKRKSGEIPCPLAVEAFAAHLSYICRY
DDKYSKYFISHKPNKTWQQVFWFAISIAINNAYILYKMSDAYHVKRYSRAQFGERLVRELLGLE
DASPTH.
The CTSG (P08311) has an amino acid sequence as follows (SEQ ID NO: 2):
MQPLLLLLAFLLPTGAEAGEIIGGRESRPHSRPYMAYLQIQSPAGQSRCGGFLVREDFVLTAA
HCWGSNINVTLGAHNIQRRENTQQHITARRAIRHPQYNQRTIQNDIMLLQLSRRVRRNRNVNPV
ALPRAQEGLRPGTLCTVAGWGRVSMRRGTDTLREVQLRVQRDRQCLRIFGSYDPRRQICVGDR
RERKAAFKGDSGGPLLCNNVAHGIVSYGKSSGVPPEVFTRVSSFLPWIRTTMRSFKLLDQMETPL.
The WARS1 (P23381) has an amino acid sequence as follows (SEQ ID NO: 3):
MPNSEPASLLELFNSIATQGELVRSLKAGNASKDEIDSAVKMLVSLKMSYKAAAGEDYKADC
PPGNPAPTSNHGPDATEAEEDFVDPWTVQTSSAKGIDYDKLIVRFGSSKIDKELINRIERATGQRP
HHFLRRGIFFSHRDMNQVLDAYENKKPFYLYTGRGPSSEAMHVGHLIPFIFTKWLQDVFNVPLVI
QMTDDEKYLWKDLTLDQAYSYAVENAKDIIACGFDINKTFIFSDLDYMGMSSGFYKNVVKIQK
HVTFNQVKGIFGFTDSDCIGKISFPAIQAAPSFSNSFPQIFRDRTDIQCLIPCAIDQDPYFRMTRDVA
PRIGYPKPALLHSTFFPALQGAQTKMSASDPNSSIFLTDTAKQIKTKVNKHAFSGGRDTIEEHRQF
GGNCDVDVSFMYLTFFLEDDDKLEQIRKDYTSGAMLTGELKKALIEVLQPLIAEHQARRKEVTD
EIVKEFMTPRKLSFDFQ
The SELL (P14151) has an amino acid sequence as follows (SEQ ID NO: 4):
MIFPWKCQSTQRDLWNIFKLWGWTMLCCDFLAHHGTDCWTYHYSEKPMNWQRARRFCRD
NYTDLVAIQNKAEIEYLEKTLPFSRSYYWIGIRKIGGIWTWVGTNKSLTEEAENWGDGEPNNKK
NKEDCVEIYIKRNKDAGKWNDDACHKLKAALCYTASCQPWSCSGHGECVEIINNYTCNCDVGY
YGPQCQFVIQCEPLEAPELGTMDCTHPLGNFSFSSQCAFSCSEGTNLTGIEETTCGPFGNWSSPEPT
CQVIQCEPLSAPDLGIMNCSHPLASFSFTSACTFICSEGTELIGKKKTICESSGIWSNPSPICQKLDKS
FSMIKEGDYNPLFIPVAVMVTAFSGLAFIIWLARRLKKGKKSKRSMNDPY
The Pro-SFTPB (P07988) has an amino acid sequence as follows (SEQ ID NO: 5):
MAESHLLQWLLLLLPTLCGPGTAAWTTSSLACAQGPEFWCQSLEQALQCRALGHCLQEVWG
HVGADDLCQECEDIVHILNKMAKEAIFQDTMRKFLEQECNVLPLKLLMPQCNQVLDDYFPLVID
YFQNQTDSNGICMHLGLCKSRQPEPEQEPGMSDPLPKPLRDPLPDPLLDKLVLPVLPGALQARPG
PHTQDLSEQQFPIPLPYCWLCRALIKRIQAMIPKGALAVAVAQVCRVVPLVAGGICQCLAERYSV
ILLDTLLGRMLPQLVCRLVLRCSMDDSAGPRSPTGEWLPRDSECHLCMSVTTQAGNSSEQAIPQA
MLQACVGSWLDREKCKQFVEQHTPQLLTLVPRGWDAHTTCQALGVCGTMSSPLQCIHSPDL.

The newly found differential biomarkers for lung cancer may be used as a candidate biomarker for differential diagnosis of lung cancer and health. One or more combinations of the biomarkers are selected to be used for an auxiliary diagnosis of lung cancer.

### Example 2: Prediction of Lung Cancer by 8 Single Biomarkers

The example used the single biomarkers screened in example 1 to establish a prediction or diagnosis model for lung cancer. The model is used to distinguish lung cancer from non-lung cancer, or to screen a patient with lung cancer from a population, or to predict whether an individual is a patient with lung cancer or the possibility of an individual suffering from lung cancer.

The ROC curve was established for each of the 8 proteins provided in example 1. An experimental result was determined by an area under the curve (AUC). The AUC of 0.5 indicated that a single protein had no diagnostic value; the AUC greater than 0.5 indicated that a single protein had a diagnostic value; and a greater AUC indicated a higher diagnostic value of the single protein. The result was shown in Table 4.

TABLE 4
ROC values for differential proteins in lung cancer and normal
healthy samples by ROC analysis and related information

| | | 95% | | | |
|---|---|---|---|---|---|
| | | confidence | Sensi- | Speci- | Critical |
| Name of biomarker | AUC | interval | tivity | ficity | value |
| PiggyBac transposable | 0.749 | 0.676-0.822 | 0.714 | 0.751 | 3.651 |
| element-derived | | | | | |
| protein 5 (PGBD5) | | | | | |
| Cathepsin G (CTSG) | 0.695 | 0.621-0.769 | 0.587 | 0.732 | 21.237 |
| Tryptophanyl-tRNA | 0.658 | 0.580-0.737 | 0.698 | 0.601 | 8.097 |
| synthetase 1 (WARS1) | | | | | |
| L-selectin (SELL) | 0.796 | 0.690-0.841 | 0.741 | 0.763 | 9.149 |
| Pro-surfactant | 0.787 | 0.717-0.857 | 0.794 | 0.685 | 50.23 |
| protein B (Pro-SFTPB) | | | | | |
| Cytokeratin 19 | 0.791 | 0.721-0.860 | 0.714 | 0.77 | 4.52 |
| fragment (Cyfra21-1) | | | | | |
| Carcinoembryonic | 0.623 | 0.544-0.701 | 0.794 | 0.408 | 4.235 |
| antigen (CEA) | | | | | |
| Cancer antigen 125 | 0.515 | 0.438-0.592 | 0.794 | 0.315 | 24.48 |
| (CA125) | | | | | |

A correlation between concentration changes of the 8 biomarkers and whether a patient suffered from lung cancer may be distinguished by the AUC values, sensitivity, and specificity in Table 4, wherein the AUC values were most visual and obvious. The higher AUC value indicated that the biomarker may more accurately distinguish a population with lung cancer and a population without lung cancer.

It can be seen from Table 4, the concentration changes of the 8 biomarkers were obviously related to whether a patient suffered from lung cancer. Any one of the 8 biomarkers was independently used, the concentration changes were used for distinguishing the population with lung cancer and the population without lung cancer, the AUC values may all reach 0.51 or more, and the biomarkers had a higher accuracy, wherein the L-selectin (SELL) had the highest correlation and the AUC value of 0.796, followed by the cytokeratin 19 fragment (Cyfra21-1) which had the AUC value of 0.791, then followed by the pro-surfactant protein B (Pro-SFTPB) which had the AUC value of 0.787, and then followed by the PiggyBac transposable element-derived protein 5 (PGBD5), the cathepsin G (CTSG), the tryptophanyl-tRNA synthetase 1 (WARS1), the carcinoembryonic antigen (CEA), and the cancer antigen 125 (CA125).

### Example 3: Classification Model for Jointly Identifying Population With Lung Cancer and Healthy Normal Population by 8 Differential Proteins, and Establishment Thereof

Although a single biomarker may also be used to distinguish serum samples of lung cancer from non-lung cancer or predict lung cancer, it is generally more accurate to combine multiple biomarkers for diagnosis or prediction.

However, after the single biomarker with a higher accuracy in predicting lung cancer was combined with other one or more biomarkers, the single biomarker did not necessarily play a larger role in the combination. At the same time, the greater number of the biomarkers did not indicate a higher prediction accuracy (AUC value) of the combination. Therefore, a large number of verification experiments were required.

The example studied a model established by 8 protein markers of the cytokeratin 19 fragment (Cyfra21-1), the carcinoembryonic antigen (CEA), the cancer antigen 125 (CA125), the pro-surfactant protein B (Pro-SFTPB), the PiggyBac transposable element-derived protein 5 (PGBD5), the cathepsin G (CTSG), the tryptophanyl-tRNA synthetase 1 (WARS1), and the L-selectin (SELL) in serums.

### 1. Data Acquisition

### Study Population:

713 cases of lung cancer and 213 cases of healthy controls were collected from August 2019 to December 2019. All enrolled patients signed an informed consent. All the patients with lung cancer were confirmed with living tissues subjected to a pathological examination, and the healthy controls were normal in a physical examination (whether the patient contains a nodule or not, or whether the patient had lung cancer or not). The enrolled people were divided, according to a ratio of 7:3, into a model group (lung cancer n=500 and healthy control n=150) and a test group (lung cancer n=213 and healthy control n=63). Data information is shown in Table 5.

TABLE 5
Information of modeled sample

| | Model group | Test group |
|---|---|---|
| Lung cancer | 500 | 213 |
| Healthy control | 150 | 63 |

Inclusion criteria for a patient with lung cancer were: (a) no history of other malignant tumors, (b) an operation treatment within one month after a blood collection, and lung cancer confirmed by a postoperative pathological examination. The healthy persons in the control group were selected from a physical examination center. These individuals were confirmed by a chest X-ray or a thin-slice computed tomography to have no lung nodules and no history of malignant tumors. After the informed consent, all the collected serum samples were stored in a serum bank at −80° C.

The example performed an enzyme-linked immunosorbent assay (ELISA) on the collected serum samples. The concentrations of the 8 protein markers of the cytokeratin 19 fragment (Cyfra21-1), the carcinoembryonic antigen (CEA), the cancer antigen 125 (CA125), the pro-surfactant protein B (Pro-SFTPB), the Piggy Bac transposable element-derived protein 5 (PGBD5), the cathepsin G (CTSG), the tryptophanyl-tRNA synthetase 1 (WARS1), and the L-selectin (SELL) in serums were obtained.

The ELSA test method was performed according to the following steps:

1. Coating: A used antigen was diluted to a proper concentration with a coating diluent (generally, the required coating amount of the antigen was 20-200 μg per well), 100 μL of the antigen was added per well and placed at 37° C. for 4 h or 4° C. for 24 h, and liquid in the well was discarded (in order to avoid evaporation, a plate should be covered with a cover or placed in a wet metal box with a wet gauze at a bottom part).

2. Blocking well of enzyme-labeling reaction: 5% of fetal bovine serum was placed at 37° C. for blocking for 40 min, each reaction well was filled with a blocking solution during the blocking, bubbles in each well were removed, and the well was washed 3 times with 3 min for each time by filling with washing liquid after the blocking was finished. The washing method was as follows: A reaction solution in the well was sucked dry, the washing liquid filled the plate well and placed for 2 min, the plate was slightly shaken, the liquid in the well was sucked dry, the liquid was poured, the plate was patted dry on an absorbent paper, and the washing was performed for 3 times:

3. Adding sample (serum) to be detected: During detection, a dilution of 1:50 to 1:400 was generally used, a larger dilution volume should be used, and a sample suction amount was generally ensured to be more than 20 μL. The diluted sample was added into the enzyme-labeling reaction well, each sample was at least added into two wells with 100 μL per well, the sample was placed at 37° C. for 40-60 min, and the washing liquid filled the well for washing for 3 times with 3 min each time.

4. Adding enzyme-labeling antibody (commercially available): The operation was performed at 37° C. for 30-60 min according to a reference working dilution degree of an enzyme conjugate provided by a provider. If the time was less than 30 min, the result was often unstable. 100 μL of the enzyme-labeling antibody was added per well and the washing was the same as before.

5. Adding substrate solution (prepared when needed): A TMB-urea hydrogen peroxide solution was first selected, followed by an OPD-hydrogen peroxide substrate solution. The substrate was added 100 μL per well, placed at 37° C. in a dark place for 3-5 min, and a stop solution was added for development.

6. Terminating reaction: 50 μL of the stop solution was added into each well to terminate the reaction and an experimental result was measured within 20 min.

7. Calculating concentration: After the OPD color development, a wavelength of 492 nm was used, and detection of a TMB reaction product required a wavelength of 450 nm. During the detection, a blank well system was first set to zero, a four-parameter Log it model was used to fit a standard curve, and the concentration of the sample was calculated.

### Statistical Analysis of Experimental Data

A test by Shapiro Wilk was used to assess a normal distribution. Differences in the concentrations of the blood markers between the patients with lung cancer and the healthy controls in the model group and the test group were respectively analyzed by using a non-parametric Wilcoxon test. In the model group, a combined diagnosis model of the 8 markers for lung cancer was constructed by using a method of combining a plurality of machine learning methods. The area under the receiver operating characteristic curve (ROC) curve (AUC) was estimated using a predicted probability value at 95% confidence interval (CI) to assess a discrimination ability of a multivariate diagnosis model. The test group was used and a Youden index (YI) was calculated to determine a predicted probability cut-off value for distinguishing the patients with lung cancer from normal controls. In addition, the ROCs for the single markers and different subgroups were constructed and compared. Standard descriptive statistic data, such as frequency, mean, median, positive predictive value (PPV), negative predictive value (NPV), and standard deviation (SD), were calculated to describe the experimental results for the study population. R3.6.1 was used for statistical analysis, and p value less than 0.05 was considered statistically significant.

### 2. Construction Steps of Lung Cancer Combined Diagnosis Model (8 MP)

S101, a concentration matrix of 8 protein markers of the cytokeratin 19 fragment (Cyfra21-1), the carcinoembryonic antigen (CEA), the cancer antigen 125 (CA125), the pro-surfactant protein B (Pro-SFTPB), the Piggy Bac transposable element-derived protein 5 (PGBD5), the cathepsin G (CTSG), the tryptophanyl-tRNA synthetase 1 (WARS1), and the L-selectin (SELL) in the samples of the model group was used as an original training data set.

S102, a generalized linear model (glmnet) algorithm was selected to be used for the construction of a prediction model and a grid search range in a hyper-parameter optimization process of the algorithm. In this step, the grid search range for the hyper-parameter optimization of a set model for each algorithm is shown in Table 6.

TABLE 6
Parameter grid search range of glmnet algorithm

| Algorithm | Parameter | Value |
|---|---|---|
| Generalized linear | alpha | 0.1, 0.55, 1 |
| model (glmnet) | lambda | 0.0003, 0.0031, 0.0311 |

S103, according to the algorithm and the hyper-parameter set range set in step S102, one hyper-parameter combination mode was selected as a constructed parameter for a prediction model.

S104, original data was divided into K subsets according to a K-fold cross-validation mechanism. To ensure that in each fold of the subsets, the ratio of majority-class samples and minority-class samples was the same as the original data set. A stratified K-fold cross-validation mechanism was used for data partitioning.

S105, according to the K training data subsets obtained by segmentation in step S104, one subset was selected as a validation set Ddev.

S106, the training data subsets which were not selected in step S105 were combined to form a training data pool Dtrainl.

S107, according to the training data set Dtrain obtained in step S106, a prediction model was constructed based on the selected supervised classification algorithm and the hyper-parameters.

S108, according to the prediction model obtained in step S107, a validation set Ddev was evaluated to obtain an AUC value, and a current prognosis prediction model and the corresponding AUC value were stored in a prediction model pool. The step S108 was the prediction model obtained according to step S107. The validation set determined in a current iteration was evaluated, and the model and the evaluation result were stored in the prediction model pool for selection and use of the subsequent prediction model. The assessment in the step may be the AUC value or other reasonable indicators for evaluating the performance of the model.

S109, whether all subsets were subjected to the validation set was determined. The step S109 was subjected to a model training to determine whether all the K subsets obtained in step S104 were used as the validation set. If all subsets were used as validation set s and the training was completed, step S110 was executed; and if there was a subset that was not used as the validation set, step S105 was performed. The step ensured that in the original data set, each sample was used as the validation set to improve model stability and prevent over-fitting of the model to a subset.

S110, the mean of the AUCs of all models of the obtained prediction model pool was used as a final performance evaluation value of the current combination mode model. The model parameters and the final performance evaluation AUC value were stored in an optimal model pool Poolbest.

S111, whether each hyper-parameter combination mode constructed the prediction model was determined. Step S111 was determining whether all the algorithms and corresponding hyper-parameter combinations obtained in step S102 were subjected to the construction of the prediction model.

If all the combination modes completed the construction of the model, step S112 was executed; and if a model was not constructed in the combination mode, step S103 was executed.

S113, a model with the largest AUC value was selected from the model set Poolbest obtained in step S112 as a final prediction model for diagnosing lung cancer.

### 3. Parameter Optimization Result of Lung Cancer Combined Diagnosis Model (8 MP)

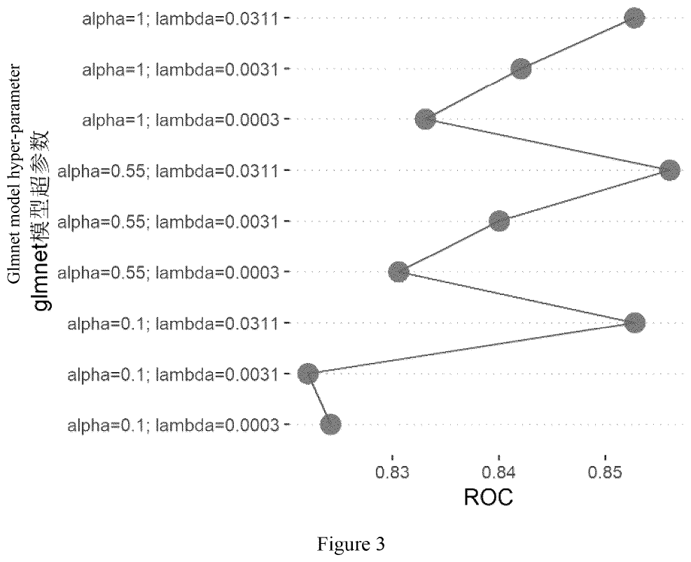

Through the execution of the model construction steps, a model (FIG. 3) constructed under the combination of the hyper-parameters of 9 different glmnet algorithms was obtained, and the performance of the model was evaluated through the AUC values. As shown in Table 7 and FIG. 3, when the glmnet algorithm hyper-parameter combination was alpha=0.55 and lambda=0.0311, the AUC reaches the maximum value 0.8561 (the AUC was calculated by using a 10-fold cross-validation method in the modeling process).

TABLE 7
AUC of model constructed under different hyper-
parameter combinations of glmnet algorithm

| ALPHA | LAMBDA | AUC |
|---|---|---|
| 0.1 | 0.0003 | 0.8241 |
| 0.1 | 0.0031 | 0.8220 |
| 0.1 | 0.0311 | 0.8528 |
| 0.55 | 0.0003 | 0.8305 |
| 0.55 | 0.0031 | 0.8400 |
| 0.55 | 0.0311 | 0.8561 |
| 1 | 0.0003 | 0.8331 |
| 1 | 0.0031 | 0.8421 |
| 1 | 0.0311 | 0.8527 |

An equation for constructing the model based on the optimal hyper-parameter combination was as follows:

Y=Σᵢ₌₁^{m}Kᵢ*Xᵢ+b

Wherein Y is a predictive value, i represents an i^{th} biomarker, m represents the number of biomarkers (m=8), Xᵢ represents a detection value (μg/mL) of the i^{th} biomarker, Kᵢ represents a coefficient of the i^{th} biomarker (Table 8), and b is a constant 3.261652.

TABLE 8
Coefficients of 8 biomarkers in model

| Biomarker | Coefficient |
|---|---|
| Cyfra21-1 | −0.76761 |
| CEA | 1 |
| CA125 | 0.434921 |
| Pro-SFTPB | −0.72697 |
| PGBD5 | −0.14199 |
| CTSG | 1 |
| WARS1 | 1 |
| SELL | 1 |

### 4. Determination of Diagnosis Threshold of Lung Cancer Combined Diagnosis Model (8 MP)

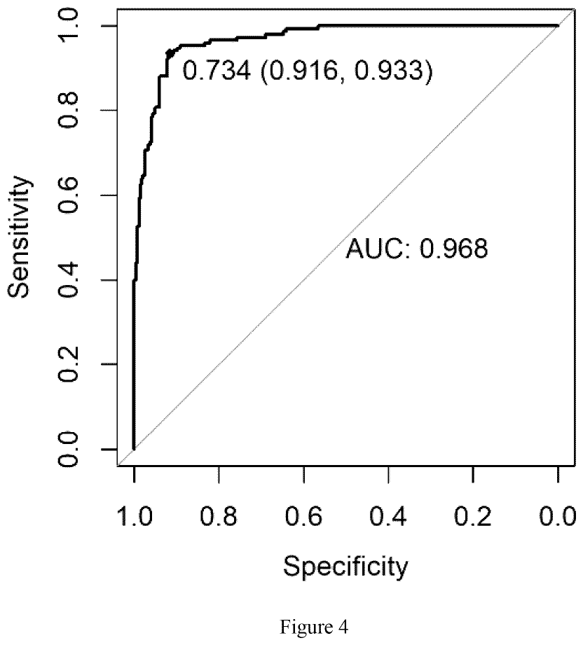

A ROC curve was plotted based on the predictive values in the model group and an optimal diagnostic cutoff value was set to be 0.734 based on the Youden index value. When the predicted value Y is less than or equal to 0.734, it is determined that the individual is not a lung cancer patient; when the predicted value Y is greater than 0.734, it is determined that the individual is a lung cancer patient. The result is shown in FIG. 4: The model in the model group had the AUC of 0.968, the sensitivity of 70.7%, and the specificity of 84.8%.

### 5. Verification of Lung Cancer Combined Diagnosis Model (8 MP)

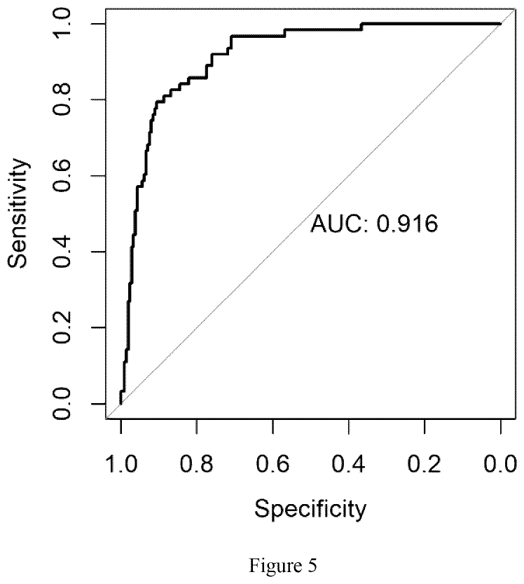

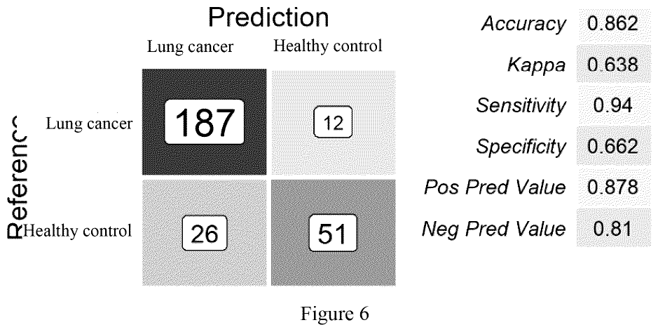

A ROC curve was plotted based on the predictive values in the test group. As shown in FIG. 5, the AUC was 0.916. Besides, the optimal diagnostic cutoff was set to be 0.734 based on the Youden index value. When the predictive value of the diagnosis model was ≤ 0.734, an individual to be tested was not considered as a patient with lung cancer; and when the predictive value of the model >0.734, an individual to be tested was considered as a patient with lung cancer. The result is shown in FIG. 6: The model in the test group had the accuracy of 86.2%, the Kappa value of 0.638, the sensitivity of 94%, the specificity of 66.2%, the positive prediction rate of 87.8%, and the negative prediction rate of 81%.

### Example 4: Comparison of Diagnostic Value of Different Lung Cancer Diagnosis Models

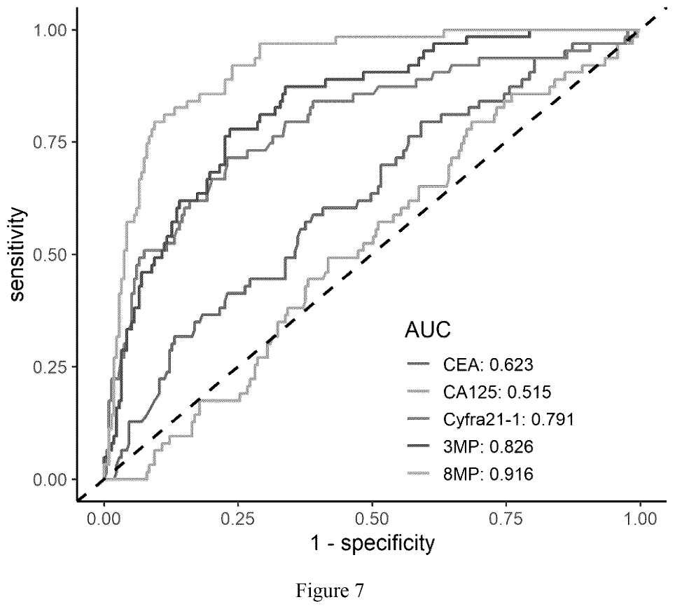

To further analyze and study a diagnostic value of the model (8 MP) provided in example 3, the performance was compared with that of traditional markers (CEA, CA125, and Cyfra21-1) and a combination thereof (3 MP, comprising CEA, CA125, and Cyfra21-1). A specific model equation was: Y=CEA-0.76761*Cyfra21-1+CEA+0.434921*CA125+CTSG−0.72697*Pro-SFTPB+WARS1−0.14199*PGBD5+SELL+3.261652). The comparison was performed in the test group. The result is shown in FIG. 7 and Table 9.

TABLE 9
Comparison of areas under ROC curves
of different diagnosis models

| PANEL | AUC | 95% Cl | DIFF | P VALUE |
|---|---|---|---|---|
| CEA | 0.623 | 0.544-0.701 | −0.292642 | 2.65E−13 |
| CA125 | 0.515 | 0.438-0.592 | −0.400642 | 3.36E−20 |
| CYFRA21-1 | 0.791 | 0.721-0.86 | −0.124642 | 1.51E−04 |
| 3MP | 0.826 | 0.771-0.882 | −0.089642 | 5.68E−04 |
| 8MP | 0.916 | 0.88-0.952 | / | / |

As shown in FIG. 7 and Table 8, the model (8 MP) had the AUC of 0.29, 0.4, and 0.12 higher than the traditional single marker, respectively, and 0.09 higher than the traditional marker combination (3 MP). An AUC difference significance test method, DeLong's test, was used. The result showed that the diagnostic value of the model (8 MP) was significant (p<0.05) higher than that of the traditional markers or the traditional marker combination model.

All the patents and publications mentioned in the description of the present disclosure indicate that these are public technologies in the art and may be used by the present disclosure. All the patents and publications cited herein are listed in the references, just as each publication is specifically referenced separately. The present disclosure described herein may be realized in the absence of any one element or multiple elements, one restriction or multiple restrictions, where the limitation is not specifically described here. For example, the terms “comprising”, “essentially consisting of”, and “consisting of” in each example herein may be replaced by the rest 2 terms. The so-called “a” here only means “a kind”, not excluding only one, but also may indicate 2 or more. The terms and expressions used herein are descriptive, without limitation. Besides, there is no intention to indicate that these terms and interpretations described in the description exclude any equivalent features. However, it may be known that any appropriate changes or modifications may be made within the scope of the present disclosure and claims. It may be understood that the examples described in the present disclosure are some preferred examples and features. A person skilled in the art may make some modifications and changes according to the essence of the description of the present disclosure. These modifications and changes are also considered to fall within the scope of the present disclosure and the scope limited by independent claims and dependent claims.

1. A method for diagnosing a presence of lung cancer in an individual, wherein the method comprises these steps:
providing a liquid sample obtained from an individual;
determining a concentration of a biomarker in the liquid sample; and
classifying the individual as either a healthy individual or an individual with lung cancer based on the determined concentration of the biomarker;
wherein the biomarker is: PGBD5, CTSG, WARS1, or SELL.

2. The method according to claim 1, wherein the liquid sample comprises any one of blood, urine, saliva or sweat.

3. The method according to claim 2, wherein the blood sample is a serum sample, a whole blood sample or a plasma sample.

4. The method according to claim 1, wherein a measurement method for determining the concentration of the biomarker includes enzyme-linked immunosorbent assay (ELISA), protein/peptide microarray detection, immunoblotting, bead-based immunoassay, or microfluidic immunoassay.

5. The method according to claim 1, wherein the biomarker further comprises Cyfra21-1, CEA, CA125, or Pro-SFTPB.

6. A method for diagnosing a presence of lung cancer in an individual, wherein the method comprises these steps:
providing a liquid sample obtained from an individual;
determining a concentration of biomarkers in the liquid sample; and
classifying the individual as either a healthy individual or an individual with lung cancer based on the determined concentration of the biomarkers;
wherein the biomarkers comprise a combination of biomarkers selected from two or more of the biomarkers as follows: PGBD5, CTSG, WARS1, and SELL.

7. The method according to claim 6, wherein the biomarkers further comprise one of biomarkers as follows: Cyfra21-1, CEA, CA125, and Pro-SFTPB.

8. The method according to claim 6, wherein the biomarkers comprise a combination of biomarkers selected from three or more of the following biomarkers: PGBD5, CTSG, WARS1, SELL, Cyfra21-1, CEA, CA125, and Pro-SFTPB.

9. The method according to claim 6, wherein the biomarkers comprise a combination of biomarkers selected from the following eight biomarkers: PGBD5, CTSG, WARS1, SELL, Cyfra21-1, CEA, CA125, and Pro-SFTPB.

10. The method according to claim 6, wherein the biomarkers consists of the following biomarkers: PGBD5, CTSG, WARS1, SELL, Cyfra21-1, CEA, CA125, and Pro-SFTPB.

11. The method according to claim 10, the method further comprises a data analysis module, wherein the data analysis module is configured to receive and analyze concentration values of the biomarkers.

12. The method according to claim11, wherein the data analysis module calculates a predictive value for determining whether an individual has lung cancer by substituting the concentration values of the biomarker into an equation, thereby evaluating the individual's likelihood of having lung cancer; wherein the equation is as follows:
Y=Σᵢ₌₁^{m}Kᵢ*Xᵢ+b
wherein Y is a predictive value, i represents an i^{th} biomarker, m represents the number of biomarkers (m=8), Xᵢ represents a concentration value of the i^{th} biomarker, Kᵢ represents a coefficient of the i^{th} biomarker, and b is a constant 3.261652; and
the coefficient Kᵢ is shown in the following table:

| Biomarker | Coefficient |
|---|---|
| Cyfra21-1 | −0.76761 |
| CEA | 1 |
| CA125 | 0.434921 |
| Pro-SFTPB | −0.72697 |
| PGBD5 | −0.14199 |
| CTSG | 1 |
| WARS1 | 1 |
| SELL | 1. |

13. The method according to claim 12, wherein when the predicted value Y is less than or equal to 0.734, it is determined that the individual is not a lung cancer patient; when the predicted value Y is greater than 0.734, it is determined that the individual is a lung cancer patient.

14. The method according to claim 10, wherein PGBD5 has an amino acid sequence with UniProt database identifier Q8N414; CTSG has an amino acid sequence with UniProt database identifier P08311; WARS1 has an amino acid sequence with UniProt database identifier P23381; SELL has an amino acid sequence with UniProt database identifier P14151; Pro-SFTPB has an amino acid sequence with UniProt database identifier P07988; CA125 has an amino acid sequence with UniProt database identifier Q8WXI7; CEA has an amino acid sequence with UniProt database identifier Q13984; Cyfra21-1 has an amino acid sequence with UniProt database identifier P08727.

15. A system for predicting whether an individual has lung cancer comprising a data analysis module configured to receive concentration values of biomarkers in a fluid sample, wherein the biomarkers consist of the following: PGBD5, CTSG, WARS1, SELL, Cyfra21-1, CEA, CA125, and Pro-SFTPB.

16. The system according to claim 15, wherein the data analysis module calculates a predictive value for determining whether an individual has lung cancer by substituting the concentration values of the biomarkers into an equation, thereby evaluating the individual's likelihood of having lung cancer, wherein the equation is as follows:
Y=Σᵢ₌₁^{m}Kᵢ*Xᵢ+b
wherein Y is a predictive value, i represents an i^{th} biomarker, m represents the number of biomarkers and is equal to 8, Xᵢ represents a concentration value of the i^{th} biomarker with a unit of μg/mL, Kᵢ represents a coefficient of the i^{th} biomarker, and b is a constant 3.261652; and
the coefficient Kᵢ is shown in the following table:

| Biomarker | Coefficient |
|---|---|
| Cyfra21-1 | −0.76761 |
| CEA | 1 |
| CA125 | 0.434921 |
| Pro-SFTPB | −0.72697 |
| PGBD5 | −0.14199 |
| CTSG | 1 |
| WARS1 | 1 |
| SELL | 1. |

17. The system according to claim 15, wherein PGBD5 has an amino acid sequence with UniProt database identifier Q8N414; CTSG has an amino acid sequence with UniProt database identifier P08311; WARS1 has an amino acid sequence with UniProt database identifier P23381; SELL has an amino acid sequence with UniProt database identifier P14151; Pro-SFTPB has an amino acid sequence with UniProt database identifier P07988; CA125 has an amino acid sequence with UniProt database identifier Q8WXI7; CEA has an amino acid sequence with UniProt database identifier Q13984; Cyfra21-1 has an amino acid sequence with UniProt database identifier P08727.

18. The system according to claim 16, wherein when the predicted value Y is less than or equal to 0.734, it is determined that the individual is not a lung cancer patient; and when the predicted value Y is greater than 0.734, it is determined that the individual is a lung cancer patient.

19. The system according to claim 15, wherein the system further includes a detection module for detecting the biomarkers, wherein the detection module comprises a kit for enzyme-linked immunosorbent assay (ELISA), protein/peptide microarray detection, immunoblotting, bead-based immunoassay, or microfluidic immunoassay.

20. The system according to claim 15, wherein the system further includes a display screen for inputting the detection results.


SEQUENCE LISTING (5 sequences)

SEQ ID NO: 1 - protein, 524 aa, unidentified
MAEGGGGARRRAPALLEAARARYESLHISDDVFGESGPDSGGNPFYSTSAASRSSSAASSDDEREPPGPPGAAPPPPRAPDAQEPEEDEAGAGWSAALRDRPPPRFEDTGGPTRKMPPSASAVDFFQLFVPDNVLKNMVVQTNMYAKKFQERFGSDGAWVEVTLTEMKAFLGYMISTSISHCESVLSIWSGGFYSNRSLALVMSQARFEKILKYFHVVAFRSSQTTHGLYKVQPFLDSLQNSFDSAFRPSQTQVLHEPLIDEDPVFIATCTERELRKRKKRKFSLWVRQCSSTGFIIQIYVHLKEGGGPDGLDALKNKPQLHSMVARSLCRNAAGKNYIIFTGPSITSLTLFEEFEKQGIYCCGLLRARKSDCTGLPLSMLTNPATPPARGQYQIKMKGNMSLICWYNKGHFRFLTNAYSPVQQGVIIKRKSGEIPCPLAVEAFAAHLSYICRYDDKYSKYFISHKPNKTWQQVFWFAISIAINNAYILYKMSDAYHVKRYSRAQFGERLVRELLGLEDASPTH

SEQ ID NO: 2 - protein, 240 aa, synthetic construct
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRT

SEQ ID NO: 3 - protein, 420 aa, unidentified
MPNSEPASLLELFNSIATQGELVRSLKAGNASKDEIDSAVKMLVSLKMSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCAIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDY

SEQ ID NO: 4 - protein, 372 aa, unidentified [sequence omitted]

SEQ ID NO: 5 - protein, 381 aa, unidentified
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL